(19) United States
(12) Patent Application Publication
(10) Pub. No.: US 2007/0246570 A1
(43) Pub. Date: Oct. 25, 2007

(54) Portable texture sprayer

(76) Inventor: Craig Cataline, Lafayette, CA (US)

Correspondence Address: John Nielsen;RANDICK O'DEA & TOOLIATOS, LLP, 5000 HOPYARD ROAD, SUITE 400, PLEASANTON, CA, 94588, US

(21) Appl. No.: 11/403,001

(22) Filed: Apr. 11, 2006

(51) Int. Cl. A61M 11/02

(52) U.S. Cl. 239/373; 239/152; 239/525; 239/320; 239/323; 239/346; 222/107; 222/175

(57) ABSTRACT

The invention comprises a handheld drywall joint compound and acoustic ceiling texture application device. An embodiment of the invention comprises an expansion bag within a tube. The tube also contains texture material, and as the bag is inflated in the tube, the texture material is forced into a spray device that is connected to the tube. The spray device allows the user to apply the texture material to the desired locations.

### I. BACKGROUND OF THE INVENTION

A. Field of Invention

This invention relates generally to an apparatus for dispensing texture coating to a surface to create a finish pattern, and in particular embodiments, this invention relates to apparatus for dispensing texture coating to finish drywall and ceiling surfaces.

B. Discussion of the Prior Art

Drywall has become a very popular material in the construction of building interiors. In particular, building interiors generally have vertical stud walls that support pre-formed drywall panels attached to the stud walls. Joints between the adjacent drywall panels are taped and finished with joint compound before painting or wallpapering. Many drywall finishing tools have been developed over the years to facilitate finishing with joint compound.

Denkins U.S. Pat. No. 2,815,142 discloses a portable drywall joint compound pump station that includes a pneumatic pump for pumping joint compound from a mud container. The device includes a frame containing four legs and a protective cage. Mills (U.S. Pat. No. 6,712,238) discloses drywall taping and texture system using a bladder pump for pumping material from a container to a work surface that includes a pump housing, a compressed air supply, a plurality of air release mechanisms, and an inflatable bladder mounted within the pump housing and held between upper and lower valves for controlling the flow of the material.

In most of the existing devices in the field, the apparatus used to spray on the drywall compound sits or rolls on the floor surface, and can be difficult to manipulate and maneuver. Also, some of the existing devices are limited to spraying on vertical surfaces, as well as having very limited capacity. Most of the existing devices in the field require the use of large compressors, and only allow compound to be one thickness. Also, many of the existing devices are difficult to load, and are limited in their ability to spray at various angles, including vertically onto ceilings.

### II. SUMMARY OF THE INVENTION

The invention comprises a portable drywall joint compound sprayer for spraying a finished coating of texture on relatively flat surfaces. Drywall joint compound, as well as other finish materials, including ceiling finish compound, capable of being applied by the invention described herein shall hereinafter be referred to as “compound” or “texture material” interchangeably. The device is a generally low-pressure sprayer that includes a compressed air powered piston that pushes the texture material to the spray nozzle. The base portion of the device allows the user to easily fill the device with compound, while also allowing for easy cleaning. The shoulder mount allows for convenient handling. The device includes air and spray controls that allow for texture integrity, a sealed container design allows the compound to stay consistent during use. The design of the device allows it to be run with a relatively small air compressor using lower pressures.

The shoulder mount design allows the operator to balance the load of the sprayer as well as carry a relatively large load of compound, and allows for better aim and control of the sprayer with less user fatigue. The portion of the tube containing the texture material is sealed to prevent the texture material from drying out while in use, as well as making it possible to transport the sprayer to multiple locations without the need for cleaning between jobs. The spray pattern can be regulated through the use of the air control lever as well as a lever to control the amount of compound that is discharged from the sprayer.

The compound is moved forward within the device via an inflatable bag located towards the rear of the tube, and behind the piston. The bag is inflated using the same air compressor that is used to spray the compound. As the user inflates and expands the bag, using the air from the compressor, the bag forces the piston towards the front of the tube. This, in turn, pushes the compound forward and towards the front of the tube. An opening at the front of the tube allows compound material to be forced down into the spray device.

In one embodiment, as the bag inflates, the texture material is pushed forward by the piston at a steady rate. The piston may have a front scoop that assists in pushing the compound forward and seals the tube, thus keeping the compound from getting behind the piston. The piston may also comprise a moisturizing sponge that assists in maintaining the desired moisture level of the compound in the tube.

Numerous other advantages and features of the invention will become readily apparent from the detailed description of the preferred embodiment of the invention, from the claims, and from the accompanying drawings, in which like numerals are employed to designate like parts throughout the same.

### III. BRIEF DESCRIPTIONS OF THE DRAWINGS

FIG. 1 is a side view of the present invention.

FIG. 2 is a front view of the present invention.

FIG. 3 is a cross section view of the present invention.

### IV. DETAILED DESCRIPTIONS OF THE DRAWINGS

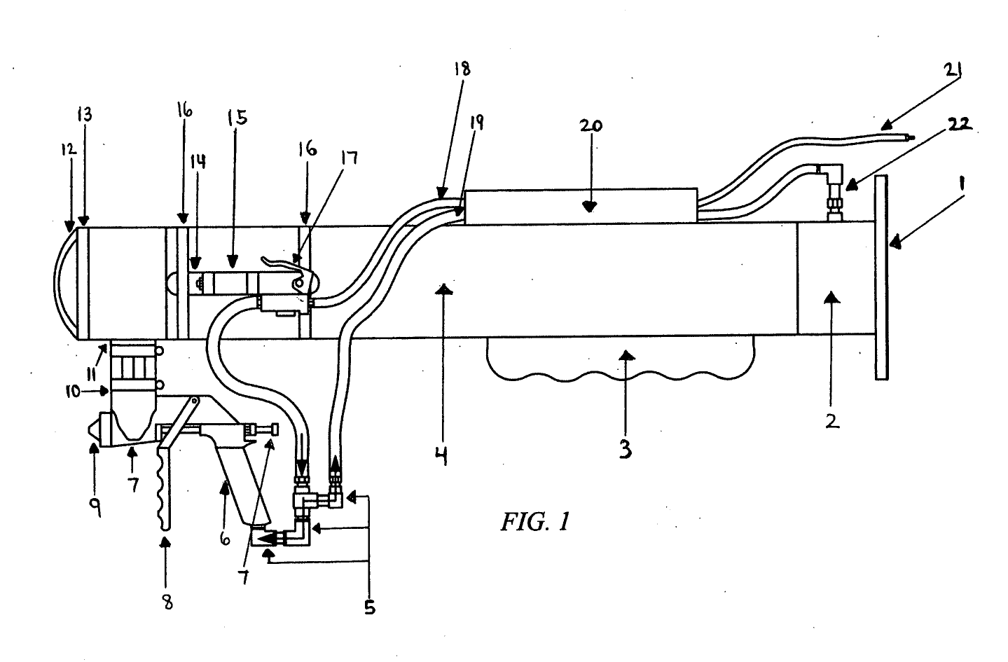

With reference now to the drawings, and in particular to FIGS. 1 through 3 thereof, a portable texture sprayer embodying the present invention is shown. The design of the present invention allows the texture to be consistent despite variations in outside temperature and humidity. The design of the present invention allows the user to point the device in any direction.

In FIG. 1, the fill stand 1 is attached (preferably threaded) to a base piece 2, and acts to secure and end of the expansion bag 23, as well as allow the device to be placed in an upright position. The fill stand 1 and base piece 2 may be made from a variety of different material, however they should preferably be made of lightweight materials such as plastic. The base piece 2, as described above, is attached to the fill stand 1, and provides an opening in the body, preferably on top of the device, that allows an attachment to the expansion bag coupler 22. A shoulder pad 3 is shown on the bottom of the device. It is envisioned that the user will position a portion of the device on their shoulder during use. As such, a shoulder pad 3 is preferably. present to increase the comfort of the user. The shoulder pad 3 is attached to the underside of the body 4 of the device.

Although the body 4 of the device can be made from a variety of materials, it is preferably made from lightweight materials such as plastic. The base 4 holds the internal parts of the device as well as the texture material before and during use.

A pneumatic pump supplies compressed air to the device. The air joints 5 connect the air supply hoses 18 and 19, as well as assist in the distribution of air throughout the system. An air joint 5 also attaches to the base of the sprayer handle 6 to assist in supplying air to the sprayer system. The air pressure control valve 17 controls the rate of airflow into the expansion bag as well as the rate of airflow into (and out of) the spray nozzle.

During normal use of the device, a relatively constant amount of air pressure enters the expansion bag, thus creating a constant pressure on the piston. This creates a constant pressure on the texture being pushed forward in the body of the device and thereafter into the handle/body connecting tube 10. The sprayer handle 6 is used to hold the device while in use.

The trigger 8 affects the amount of air allowed to flow through the nozzle 9, as well as the amount of texture material allowed to enter the sprayer chamber 7, and thus the amount of material sprayed from the device. When the trigger 8 is pulled back, this pulls back a tube that was obstructing the nozzle 9 opening. Pulling the trigger 8 back also creates an opening from the handle/body connecting tube 10, thus allowing texture material to enter the sprayer chamber 7. As the texture enters the chamber 7, compressed air propels it out the nozzle 9, and onto the desired location.

The handle/body connecting tube 10 is preferably attached to the body 4 of the device at a connecting joint 11, preferably located near the front end of the body 4. The fill cap 13 is attached to the front of the body 4, preferably by screwing it in. The fill cap 13 has a fill cap handle 12 to assist in this action. This allows the user to remove the fill cap 13, and then introduce the texture into the body of the device (preferably while the expansion bag is deflated). After the texture material is placed in the body 4, the user then re-attaches the fill cap 13. This arrangement creates a sealable opening in the device that allows for insertion and/or removal of texture/compound material.

The air pressure control handle 14 is preferably attached to the body 4 by the handle straps 16. However alternatively it could be attached in several different ways, none of which significantly affect the use or composition of the device. The air pressure control valve 16 is attached to the control handle and allows the user to adjust the amount of air that is added to the spray nozzle in order to create the appropriate spray pattern. The air pressure control valve lever 17 is part of the control valve 15, and allows the user to control the pressure release, and the spray pattern coming out of the device.

The air supply hose 18 to the air control valve provides the conduit for the compressed air from the compressor to the air pressure control valve and the sprayer device. The air supply hose 19 to the air expansion bag provides a conduit for air to travel from the compressor to the air joints 5, and then to the expansion bag.

The air hose cover 20 keeps the air hoses 18 and 19 organized and in place. The air hose coupler 21 located on the end of the intake air supply hose 18 attaches to the air compressor. The expansion bag coupler 22, located on the end of the air supply hose 19 to the expansion bag, connects the hose to the body 4 and the expansion bag within the body 4 of the device. This expansion bag coupler 22 allows for removal of the expansion bag for cleaning, repair, replacement, or other purposes.

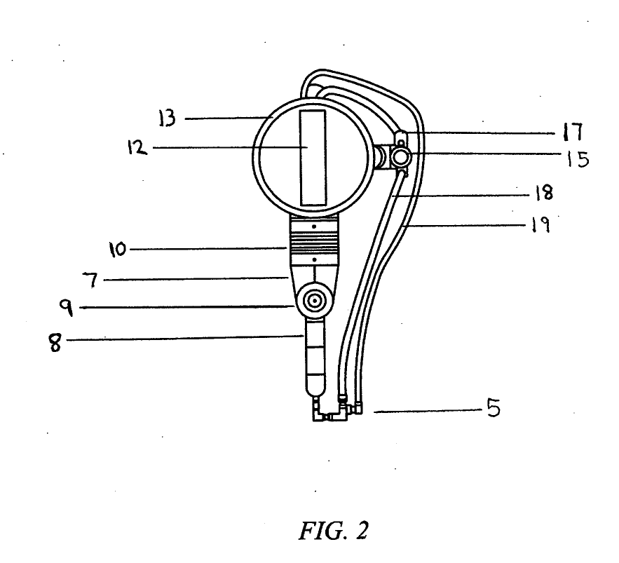

FIG. 2 is a front view of the invention. The trigger 8 is shown, as well as the front of the nozzle 9. As the user pulls the trigger 8 of the device, texture material from the chamber 7 is forced out through the nozzle 9 and onto the desired location. The front side of the fill cap 13 and fill cap handle 12 is shown, that allows the user to unscrew the cap to allow for filling the unit, as well as for cleaning purposes. The fill cap 13 should then be screwed back on prior to use. The air pressure control valve 15 and lever 17 is shown attached to the air supply hose 18 from the air compressor. The air supply hose 19 from the air joints 5 to the expansion bag coupler 22 is also shown.

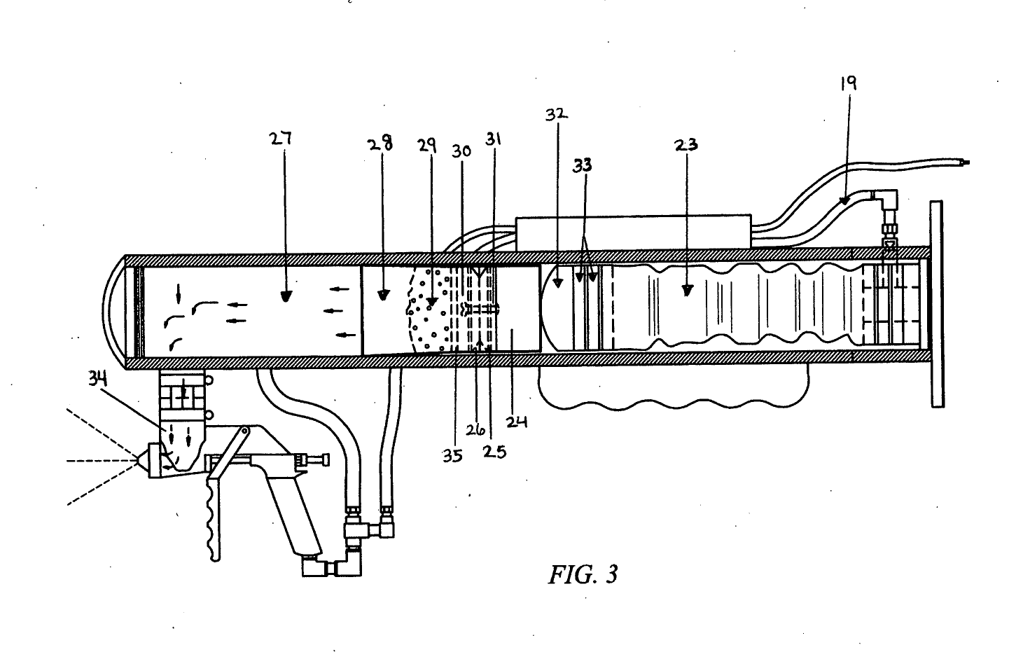

FIG. 3 is a cross section of the present invention. As the expansion bag 23 is inflated via the air supply hose 19, the piston is pushed forward. The piston as represented comprises the piston guide 24, the upper and lower pressure plates 25 and 26 respectively, the tightening bolt 30, the hydration sponge 29, and the scraper guide 28. This action of the expansion bag pushing the piston forward through the body 4 of the device pushes the texture material 27 forward and then down through the chamber, keeping a steady supply of texture material flowing into the chamber. This creates a consistent spray pattern on the target surface. The expansion bag cap 32 presses against the piston guide (24). The piston guide 24 comprises a generally circular shaped piece that keeps the piston from binding in the body 4. The pressure bolt 31 is used to hold the upper 25 and lower 26 pressure plates together, as well as holding the base of the scraper guide 28 in place. The lower pressure plate 26 fits, preferably tightly, in the interior of the body 4 as a guide for the piston. The upper pressure plate 25 is preferably slightly larger than the lower pressure plate 26, which allows for a tighter fit in the interior of the body 4. The tightening nut 30 on the pressure bolt is used to adjust the pressure on the upper and lower pressure plates. This allows the piston, which has an adjustable width, to more effectively scrape the texture material 27 from the interior of the body of the device.

The hydration sponge base 35 is preferably of a size that that creates constant outward pressure on the scraper guide 28, so as to create and maintain an outward flair of the scraper guide 28 within the tube or body 4. The hydration sponge 29 is attached to the hydration sponge base 35, and maintains the texture material at a constant moisture level, thus keeping the material from drying out. The hydration sponge 29 also keeps the interior of the tube lubricated. Also, because the hydration sponge 29 is nestled in the scraper guide 28, the moisture within the sponge expands the sponge as well as the scraper guide 28, thus shaping the guide into an outward flaring (towards the front) cone-like shape, which allows the scraper to keep better contact with the interior of the tube.

What is claimed is:

1. A portable drywall compound application device comprising:
a. a generally cylindrically shaped base unit comprising:
i. a sealable opening in the base that allows for insertion of texture material;
ii. an inflatable bag located within the base unit; and
iii. an outlet in the base unit that allows texture material within the base to flow out of the base and into a spray device;
b. a spray device comprising:
i. an intake tube from the base to accept texture material from the base unit; and
ii. a trigger mechanism for controlling the supply of compressed air to the sprayer;
c. A means for delivering compressed air from a pneumatic pump into the inflatable bag in the base unit and into the spray device.

2. The portable drywall compound application device of claim 1, further comprising:
a. a piston located proximate to the inflatable bag.

3. The portable drywall compound application device of claim 1, further comprising a means for hydrating the interior of the device.

4. The portable drywall compound application device of claim 3, wherein the means for hydrating the interior of the device comprises a sponge capable of holding liquid.

5. The portable drywall compound application device of claim 1, wherein the means for delivering compressed air from a pneumatic pump into the inflatable bag in the base unit and into the spray device comprises flexible tubing.

6. The portable drywall compound application device of claim 1, further comprising:
a. A shoulder pad located on the base unit.
